(19) United States
(12) Patent Application Publication
(10) Pub. No.: US 2014/0371742 A1
(43) Pub. Date: Dec. 18, 2014

(54) Anatomical Ablation System for the Purpose of Pulmonary Vein Isolation

(71) Applicant: VascoMed GmbH, Binzen (DE)

(72) Inventors: Bjoern Fruehauf, Berlin (DE); Michael Gellhaus, Berlin (DE); Daniele Spinelli, Cava de 'Tirreni (SA) (IT)

(21) Appl. No.: 14/299,831

(22) Filed: Jun. 9, 2014

Related U.S. Application Data
(60) Provisional application No. 61/836,172, filed on Jun. 18, 2013.

(51) Int. Cl. A61B 18/14

(52) U.S. Cl. CPC A61B 18/1492; A61B 2018/00404
USPC 606/41

(57) ABSTRACT

An ablation catheter arrangement for high-frequency ablation of tissue portions on the wall of a hollow organ or vessel in a patient, in particular for pulmonary vein isolation, Including an elongate flexible catheter body including an ablation catheter and an electrode feed line and a rinse duct in the catheter body, and also an ablation electrode and a rinse opening at a distal end of the catheter body, an advance control means for advancing or withdrawing the distal end of the ablation catheter relative to the wall of the hollow organ or vessel, and a guide path device for forcibly guiding the distal end of the ablation catheter along a curved three-dimensional movement path during advance and withdrawal.

### CROSS-REFERENCE TO RELATED APPLICATIONS

This patent application claims the benefit of co-pending U.S. Provisional Patent Application No. 61/836,172, filed on Jun. 18, 2013, the disclosure of which is hereby incorporated by reference in its entirety.

### TECHNICAL FIELD

The present invention relates to an ablation catheter arrangement for high-frequency ablation of tissue portions on the wall of a hollow organ or vessel in a patient and, in particular, for pulmonary vein isolation.

### BACKGROUND

As is known in the art, some cardiac arrhythmias can be treated by locally destroying heart tissue that acts as a (incorrect) timer or that is part of a vessel for such undesired pulses. For this purpose, different types of catheter arrangements to be used on the heart have been developed and are referred to as ablation catheter arrangements or as ablation catheters for short. Most of these arrangements, which are also used clinically, operate with high-frequency (“HF”) current, which is fed via electrodes at the end of the catheter into the tissue to be ablated and destroys said tissue by means of the resultant heating. This method has proven its worth particularly in the case of therapy of atrial fibrillation, and specifically in younger patients.

Methods and catheter arrangements that use other energy sources, for example, laser radiation, ultrasound or a coolant, with which the tissue to be ablated is destroyed by supercooling instead of heating, have also been developed. These methods and catheter arrangements, however, have previously only achieved low clinical importance, however, compared to HF ablation.

Circumferential or linear pulmonary vein isolation is a minimally invasive electrosurgical method for the treatment of idiopathic or paroxysmal (occurring in the form of sudden attacks) atrial fibrillation. Through a venous blood vessel in the groin, a catheter is inserted via the venae cavae into the right atrium of the heart and is placed in the left atrium of the heart through the cardiac septum. From here, the pulmonary vein walls are scanned using a measurement catheter, and a three-dimensional reconstruction, or what is known as a map, is created using a computer. The muscles of the pulmonary veins close to the atrium are then cauterized by means of high-frequency current catheter ablation in order to prevent the myoelectrical pulses from being transmitted to the atrium. In contrast to drug therapy, in which case the sensitivity of the atrium cells and therefore the pulse transmission is only reduced temporarily, pulmonary vein isolation is permanent. It has been demonstrated that patients with atrial fibrillation, after catheter ablation, had a significantly lower risk for Alzheimer's disease and dementia compared to patients treated by means of drug therapy.

The described intervention is time-intensive with known catheter arrangements and requires much experience on the part of the operator. In addition, second interventions are often necessary after a few months in order to close any remaining gaps in the ablation lines.

The present invention is directed toward overcoming one or more of the above-identified problems.

### SUMMARY

An object of the present invention is therefore to specify an ablation catheter arrangement well suited for pulmonary vein isolation, which in particular places few demands on the experience of the operator and enables a reduction of the intervention time and, as a result of the use thereof, makes second interventions and the need for three-dimensional computer-assisted reconstruction largely unnecessary.

At least this object is achieved by an ablation catheter arrangement having the features in independent claim 1. Expedient developments of the inventive concept are disclosed in the dependent claims.

The present invention includes the concept of assisting the operator by means of a suitable constructional design of the ablation catheter arrangement whilst maintaining an expedient ablation trail on the wall of the pulmonary vein. It also includes the concept of providing, for this purpose, a forced guidance means for the distal end of the catheter. The present invention also includes the concept of an easily handled advance control means for advancing the end of the catheter along the desired trail.

Specifically, in the case of ablation catheters having a rinse duct, which is in principle to be considered advantageous in the interest of a precise delimitation of the ablation points by means of selective cooling after the application of heat, the mechanical design is complex. The additional integration of movement path control means in the body of the ablation catheter encounters problems here for which the present invention provides a particularly advantageous solution. The assignment of a forced guidance means and advance control means arranged outside the ablation catheter avoids an additional complication of the design of the ablation catheter and the inevitably associated cost and reliability problems. In addition, the proposed solution provides greater versatility in terms of the formation of catheter arrangements for differentiated purposes and usage conditions.

To provide an outwardly closed and easily handled catheter arrangement, the ablation catheter, which may by all means be a commercially available type, and the guide path device can be housed together in an elongate, flexible main body.

In one embodiment of the present invention, the guide path device is designed to guide the distal end of the ablation catheter along a spirally or helically curved movement path. This embodiment corresponds largely to the anatomical conditions at the primary site of use, specifically at the transition of the left atrium into the pulmonary vein. Embodiments for other uses can be characterized by differently curved preferred movement paths and correspondingly configured guide path devices without hereby abandoning the concept of the present invention, as will be appreciated by one skilled in the art.

In further embodiments of the present invention, the guide path device, during use, comprises a three-dimensionally curved guide wire or a three-dimensionally curved guide sleeve, over which or into which the distal end of the ablation catheter slides during insertion and withdrawal. In accordance with the previously mentioned currently preferred geometric configuration of the movement path, the guide wire or the guide sleeve is also curved, in particular in a spiraled or helical manner, however, other geometric configurations are also possible for other fields of use.

In accordance with a further embodiment, the guide path device comprises a curvature portion formed from a shape-memory alloy, which adopts its functional shape after insertion into the hollow organ or vessel. This embodiment advantageously makes it possible to easily use and configure the guide path device at the site of use without complicated handling procedures. Other means are also available for configuration of said guide path device however, such as specific insertion sleeves or wires, from which the guide path device is released at the site of use and thereupon adopts the desired form. Additional aids of this type may also be expedient with use of a shape-memory material, at least for withdrawing the ablation catheter arrangement once an intervention has been completed.

In accordance with a further embodiment, the guide path device is assigned adjustment means that are actuatable from outside the body in order to adjust the movement path. The ablation trail to be followed by the catheter tip can hereby advantageously be changed in accordance with the specific anatomical conditions in a patient without having to produce respective patient-specific guide path devices. As a result, the likelihood of success of the pulmonary vein isolation is hereby achieved with simultaneously reduced likelihood of a second intervention.

In this case, the adjustment means can be designed, in particular to change the lateral extent and/or lateral deflection of a spiraled or helical movement path, in particular of a spirally or helically curved guide wire or of a guide sleeve. These embodiments involve less of a change to the basic shape of the catheter movement path (and therefore ablation trail), but predominantly involve the adaptation to different, patient-specific internal dimensions of the left atrium or entry of the pulmonary vein whilst simultaneously ensuring a sufficient contact pressure.

In a further embodiment, the adjustment means comprise an adjustable port with a slide mechanism for laterally displacing a through-opening for the ablation catheter or the guide wire or the guide sleeve in a port body. In this embodiment, construction elements that are known per se are used to form the ablation catheter arrangement according to the present invention, thus reducing the constructional effort and the production costs. Specifically, in this case, the adjustment means may have an adjustable port with slide mechanism of a mapping catheter arrangement, as is produced and marketed for example by the Applicant under the product name “LEXX”.

Further features, aspects, objects, advantages, and possible applications of the present invention will become apparent from a study of the exemplary embodiments and examples described below, in combination with the Figures, and the appended claims.

### DESCRIPTION OF THE DRAWINGS

Advantages and expedient aspects of the present invention will also emerge from the following description of features and exemplary embodiments of the present invention with reference to the Figures, in which:

FIG. 1 shows a schematic illustration of a catheter arrangement according to a first embodiment of the present invention, and

FIG. 2 shows a schematic illustration of a catheter arrangement according to a second embodiment of the present invention.

### DETAILED DESCRIPTION

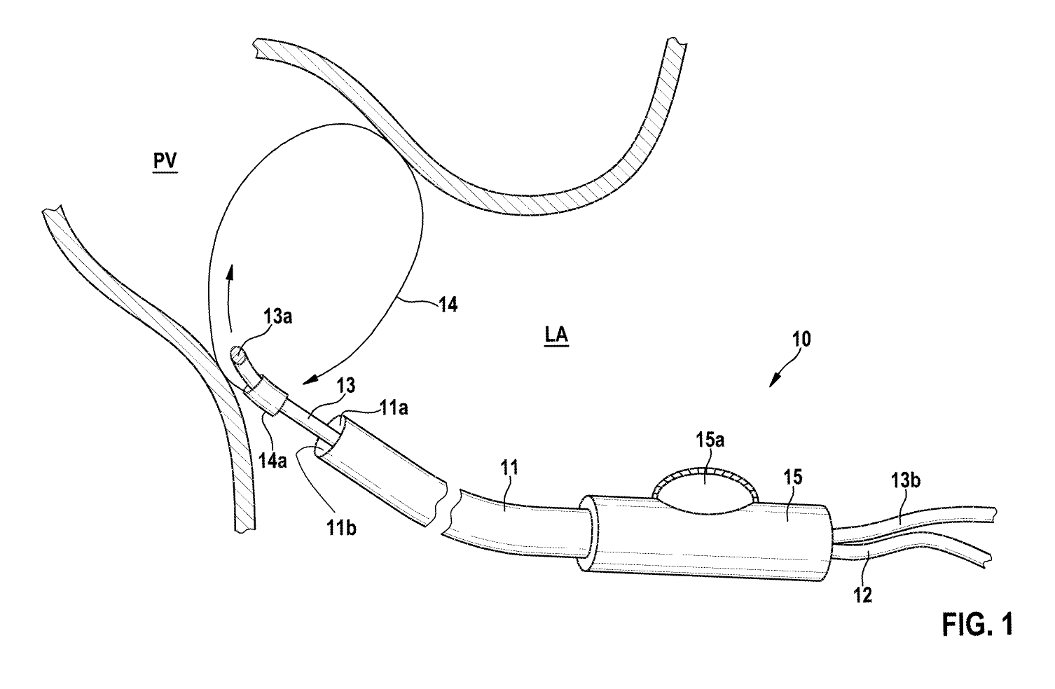

FIG. 1 shows a schematic view of a catheter arrangement 10 for pulmonary vein isolation, that is to say for ablation of vessels in the wall of the pulmonary vein “PV” leading form the left atrium “LA”. The ablation catheter arrangement 10 comprises an elongate, flexible catheter body 11 comprising a rinse duct 11b, by means of which a rinse liquid feed line 12 is connected at the proximal end of the catheter, and also comprising an ablation catheter 13, which comprises an ablation electrode 13a at the distal end and an electrode feed line 13b at the proximal end.

In the simplified illustration, the distal end 11a of the catheter body 11 is also to be understood as a rinse opening for rinsing the treatment site at the exit of the pulmonary vein PV. From the distal end 11a of the catheter body 11, an approximately circularly curved ablation guide wire 14 protrudes from the catheter body in such a way that it bears against the inner wall of the pulmonary vein PV along the entire circumference. The distal end of the ablation catheter 13 is slidably connected to the ablation electrode 13a by means of a slide piece 14a and slides forwards along the ablation guide wire 14 when an advance is implemented via an advance control wheel 15a on a grip 15 at the proximal end of the ablation catheter arrangement 10.

The distal end of the ablation catheter 13 is forcibly guided along an approximately circular to slightly helical three-dimensional movement path along the wall of the ostium or antrum. After a small advance distance, which can be adjusted precisely via the adjustment wheel 15a, the ablation electrode 13a therefore reliably reaches a next point on the vein wall at which an ablation pulse is applied, and a further point of the wall can hereby be cauterized precisely. This will normally occur as the ablation catheter 13 is advanced from the catheter body 11, however, it is also possible in principle for the distal end of the ablation catheter 13 to first advance as far as the end of the ablation guide wire 14 and to perform the sequential ablation of adjacent wall regions during the withdrawal process.

The ablation guide wire 14 preferably has a certain spring resilience and, therefore, it bears resiliently against the organ wall in the respective anatomical configuration when slid out from the catheter body 11. It may also consist of a shape-memory material and, therefore, it only adopts at body temperature, in situ, the shape shown and described in terms of function. Besides the shown forms of the ablation guide wire 14, other predetermined forms of the ablation guide wire 14 can also be implemented expediently, in particular, and for example, mixed forms from a helical and spiral curvature, which are advantageously adapted to the anatomical conditions at the transition between the left atrium and the pulmonary vein (or with adaptation at another hollow organ to the anatomical conditions in any organ).

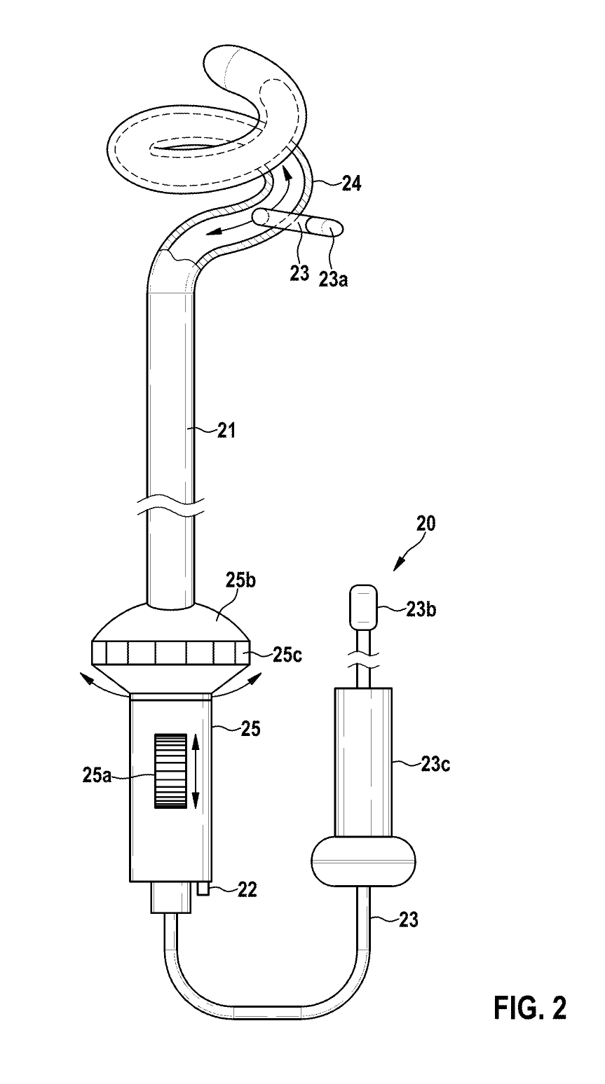

As a further exemplary embodiment, FIG. 2 shows a schematic view of an ablation catheter arrangement 20, of which the design is partially similar to the ablation catheter arrangement 10 according to FIG. 1. Like or functionally like parts are therefore denoted by reference numerals based on FIG. 1 and will not be described again hereinafter.

A key deviation lies in the fact that, in the case of the ablation catheter arrangement 20, a distal end portion of the catheter body 21 is also formed as a (adjustable) guide sleeve 24 for the distal end of the ablation catheter 23. As can be seen in FIG. 2, the distal end of the ablation catheter 23 and, therefore, the ablation electrode 23a, protrudes at an adjustable position from the wall of the guide sleeve 24 to predefine the movement path of the ablation electrode 23a. Ablation pulses can thus in turn be applied at selectable points of a predetermined movement path, and cauterization regions in a hollow organ (specifically, the pulmonary vein for example) can therefore be established precisely.

The ablation catheter 23 is advanced along the predefined movement path again by means of an adjustment wheel 25a as an advance control means, wherein, in the embodiment shown here, an additional grip 23c is provided at the proximal end of the ablation catheter 23, by means of which an advance of the ablation catheter 23 can likewise be implemented.

The grip 25 here comprises an adjustable port device 25b with a second adjustment wheel 25c, whereby the geometric configuration of the catheter end, that is to say of the guide sleeve 24, can be preset in accordance with the anatomical conditions in a specific patient. Conversely to the embodiment according to FIG. 1, where the ablation guide wire bears automatically by means of its spring resilience against the wall of the hollow organ (e.g., pulmonary vein or the like), a corresponding adjustment, that is to say in the case of the ablation catheter arrangement 20 according to FIG. 2, is performed manually by the operator. This can be advantageous when working with problematic anatomical conditions.

The operating principle of the associated adjustment device can be presumed as known per se; simple adjustment mechanisms with similar function are known for example from U.S. Pat. No. 4,960,134 and/or U.S. Pat. No. 4,777,955.

The embodiment of the present invention is not limited to these examples and the above-mentioned features, but instead a large number of modifications lying within the scope of routine activity in the art are also possible as will be appreciated by one skilled in the art.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. An ablation catheter arrangement for high-frequency ablation of tissue portions on the wall of a hollow organ or vessel in a patient for pulmonary vein isolation, the ablation catheter arrangement comprising:
an elongate flexible catheter body comprising an ablation catheter and an electrode feed line and a rinse duct in the catheter body, and also an ablation electrode and a rinse opening at a distal end of the catheter body;
an advance control means for advancing or withdrawing the distal end of the ablation catheter relative to the wall of the hollow organ or vessel; and
a guide path device for forcibly guiding the distal end of the ablation catheter along a curved three-dimensional movement path during advance and withdrawal.

2. The ablation catheter arrangement as claimed in claim 1, wherein the guide path device is arranged outside the ablation catheter and is detachable therefrom.

3. The ablation catheter arrangement as claimed in claim 1, wherein the guide path device is designed to guide the distal end of the ablation catheter along a spirally or helically curved movement path.

4. The ablation catheter arrangement as claimed in claim 1, wherein the ablation catheter and the guide path device are received together, at least to a significant extent, in the catheter body.

5. The ablation catheter arrangement as claimed in claim 1, wherein the guide path device, during use, has a three-dimensionally curved guide wire, or a three-dimensionally curved guide sleeve, over/into which the distal end of the ablation catheter slides during advance and withdrawal.

6. The ablation catheter arrangement as claimed in claim 5, wherein the three-dimensionally curved guide wire is circularly, spirally or helically curved, and wherein the three-dimensionally curved guide sleeve is circularly, spirally or helically curved.

7. The ablation catheter arrangement as claimed in claim 1, wherein the guide path device comprises a curvature portion formed from a shape-memory alloy, which adopts its functional shape after insertion into the hollow organ or vessel.

8. The ablation catheter arrangement as claimed in claim 1, wherein the guide path device includes adjustment means that are actuatable from outside the body in order to adjust the movement path.

9. The ablation catheter arrangement as claimed in claim 8, wherein the adjustment means are designed to change the lateral extent and/or lateral deflection of a circular, spiraled or helical movement path of a circularly, spirally or helically curved guide wire or of a guide wire.

10. The ablation catheter arrangement as claimed in claim 9, wherein the adjustment means comprise an adjustable port with slide mechanism for laterally displacing a through-opening for the ablation catheter or the guide wire or the guide sleeve in a port body.
